(19) United States
(12) Patent Application Publication
(10) Pub. No.: US 2006/0253199 A1
(43) Pub. Date: Nov. 9, 2006

(54) Lordosis creating nucleus replacement method and apparatus

(75) Inventors: Jean-Charles Lehuec, Bordeaux (FR); John Sherman, Wayzata, MN (US)

Correspondence Address: FAEGRE & BENSON LLP;PATENT DOCKETING, 2200 WELLS FARGO CENTER, MINNEAPOLIS, MN, 55402, US

(73) Assignee: Disc Dynamics, Inc., Eden Prairie, MN (US)

(21) Appl. No.: 11/268,856

(22) Filed: Nov. 8, 2005

Related U.S. Application Data
(60) Provisional application No. 60/708,244, filed on Aug. 15, 2005.
(60) Provisional application No. 60/677,273, filed on May 3, 2005.
(60) Provisional application No. 60/708,245, filed on Aug. 15, 2005.

(51) Int. Cl. A61F 2/44

(52) U.S. Cl. 623/17.12; 623/17.16

(57) ABSTRACT

A method of implanting an intervertebral prosthesis in a disc located between a pair of adjacent vertebrae of a patient. Damaged or diseased nucleus pulpous is removed from the disc using minimally invasive techniques. The adjacent vertebrae are positioned in a lordotic condition. A mold adapted to contain a biomaterial is positioned between the adjacent vertebrae. A flowable biomaterial is delivered into the mold using minimally invasive techniques so that the adjacent vertebrae are in the lordotic condition. The flowable biomaterial is allowed to at least partially cure so that the adjacent vertebrae are in a lordotic-neutral position. The step of positioning the pair of adjacent vertebrae in a lordotic condition may include positioning the patient in extension, displacing spinous processes of the adjacent vertebrae to a compressed configuration, suturing spinous processes of the adjacent vertebrae to a compressed configuration, and/or delivering the flowable biomaterial into the mold at sufficient pressure to distraction the adjacent vertebrae to a lordotic position. One or more preformed prostheses can be substituted for, or combined with, the mold.

The present application claims the benefit of U.S. Provisional Application Ser. No. 60/708,244 entitled Multi-Lumen Mold For Intervertebral Prosthesis And Method Of Using Same filed on Aug. 15, 2005; U.S. Provisional Application Ser. No. 60/677,273 entitled Catheter Holder for Spinal Implants filed May 3, 2005; and U.S. Provisional Application Ser. No. 60/708,245 entitled Catheter Holder for Spinal Implants filed Aug. 15, 2005, all of which are hereby incorporated by reference.

### FIELD OF THE INVENTION

The present invention relates to a method and apparatus for filling an intervertebral disc space with an in situ curable biomaterial to position a pair of adjacent vertebrae in a lordotic condition.

### BACKGROUND OF THE INVENTION

The intervertebral discs, which are located between adjacent vertebrae in the spine, provide structural support for the spine as well as the distribution of forces exerted on the spinal column. An intervertebral disc consists of three major components: cartilage endplates, nucleus pulposus, and annulus fibrosus. The central portion, the nucleus pulposus or nucleus, is relatively soft and gelatinous; being composed of about 70 to 90% water. The nucleus pulposus has a high proteoglycan content and contains a significant amount of Type II collagen and chondrocytes. Surrounding the nucleus is the annulus fibrosus, which has a more rigid consistency and contains an organized fibrous network of approximately 40% Type I collagen, 60% Type II collagen, and fibroblasts. The annular portion serves to provide peripheral mechanical support to the disc, afford torsion resistance, and contain the softer nucleus while resisting its hydrostatic pressure.

Intervertebral discs, however, are susceptible to disease and a number of injuries. Disc herniation occurs when the nucleus begins to extrude through an opening in the annulus, often to the extent that the herniated material impinges on nerve roots in the spine or spinal cord. The posterior and posterolateral portions of the annulus are most susceptible to attenuation or herniation, and therefore, are more vulnerable to hydrostatic pressures exerted by vertical compressive forces on the intervertebral disc. Various injuries and deterioration of the intervertebral disc and annulus fibrosus are discussed by Osti et al., Annular Tears and Disc Degeneration in the Lumbar Spine, J. Bone and Joint Surgery, 74-B(5), (1982) pp. 678-682; Osti et al., Annulus Tears and Intervertebral Disc Degeneration, Spine, 15(8) (1990) pp. 762-767; Kamblin et al., Development of Degenerative Spondylosis of the Lumbar Spine after Partial Discectomy, Spine, 20(5) (1995) pp. 599-607.

Many treatments for intervertebral disc injury have involved the use of nuclear prostheses or disc spacers. A variety of prosthetic nuclear implants are known in the art. For example, U.S. Pat. No. 5,047,055 (Bao et al.) teaches a swellable hydrogel prosthetic nucleus. Other devices known in the art, such as intervertebral spacers, use wedges between vertebrae to reduce the pressure exerted on the disc by the spine. Intervertebral disc implants for spinal fusion are known in the art as well, such as disclosed in U.S. Pat. No. 5,425,772 (Brantigan) and U.S. Pat. No. 4,834,757 (Brantigan).

Further approaches are directed toward fusion of the adjacent vertebrate, e.g., using a cage in the manner provided by Sulzer. Sulzer's BAK® Interbody Fusion System involves the use of hollow, threaded cylinders that are implanted between two or more vertebrae. The implants are packed with bone graft to facilitate the growth of vertebral bone. Fusion is achieved when adjoining vertebrae grow together through and around the implants, resulting in stabilization.

Prosthetic implants formed of biomaterials that can be delivered and cured in situ, using minimally invasive techniques to form a prosthetic nucleus within an intervertebral disc have been described in U.S. Pat. No. 5,556,429 (Felt) and U.S. Pat. No. 5,888,220 (Felt et al.), and U.S. Patent Publication No. US 2003/0195628 (Felt et al.), the disclosures of which are incorporated herein by reference. The disclosed method includes, for instance, the steps of inserting a collapsed mold apparatus (which in a preferred embodiment is described as a “mold”) through an opening within the annulus, and filling the mold to the point that the mold material expands with a flowable biomaterial that is adapted to cure in situ and provide a permanent disc replacement. Related methods are disclosed in U.S. Pat. No. 6,224,630 (Bao et al.), entitled “Implantable Tissue Repair Device” and U.S. Pat. No. 6,079,868 (Rydell), entitled “Static Mixer”, the disclosures of which are incorporated herein by reference. See also, for instance, French Patent Appl. No. FR 2 639 823 (Garcia) and U.S. Pat. No. 6,187,048 (Milner et al.). Both references differ in several significant respects from each other and from the apparatus and method described below.

Nucleoplasty or partial disc replacement performed from posterior entry points have a high rate of dislocation, often due to the fact that the posterior wall of the annulus is thinner than the other walls, and may be diseased or damaged. While anterior entry points are often appropriate for many patients, the posterior approach is the most desirable for a large segment of the patient population.

As illustrated in FIGS. 1 and 2, dislocation of intervertebral disc prostheses 20 can occur due to expulsion forces 22, 24 generated during flexion or rotation of the adjacent vertebrae 28, 30. The expulsion forces 22, 24 are created by opposing end plates 42, 44 of the adjacent vertebrae 28, 30 acting on the prosthesis 20 at angle 26. The greater the angle 26, the greater the expulsion forces 22, 24.

The posterior wall 32 of the annulus 34 is typically thinner than the other walls, and may include damaged or diseased portions 46. Damage to the posterior wall 32 can be aggravated during the surgical removal of the nucleus pulposus 36. For example, each annulotomy 40 through the annulus 34 further weakens the posterior wall 32, unless the annulotomy is positioned through a herniation site. Also, in situations where the size of the prosthesis 20 is small relative to the size of the annulotomy 40, the prosthesis 20 can extrude posteriorly 38 from the annulus 34. If dislocation occurs, the prosthesis 20 and/or portions of the annulus 34 can impinge on the spinal cord or nerve root, causing pain and other complications.

### BRIEF SUMMARY OF THE INVENTION

The present invention relates to a method and apparatus for positioning a pair of adjacent vertebrae in a lordotic condition. The lordotic condition is primarily anterior distraction of a pair of adjacent vertebrae that does not cause symptomatic impingement of the spinal cord by the posterior portion of the intervertebral disc. In the preferred embodiment, the method includes delivering an in situ curable biomaterial to the intervertebral disc space.

The present method and apparatus can be used, for example, to implant a prosthetic total disc, or a prosthetic disc nucleus, using minimally invasive techniques that leave the surrounding disc tissue substantially intact. The phrase intervertebral disc prosthesis is used generically to refer to both of these variations.

Minimally invasive refers to a surgical mechanism, such as microsurgical, percutaneous, or endoscopic or arthroscopic surgical mechanism, that can be accomplished with minimal disruption of the pertinent musculature, for instance, without the need for open access to the tissue injury site or through minimal incisions (e.g., incisions of less than about 4 cm and preferably less than about 2 cm). Such surgical mechanism are typically accomplished by the use of visualization such as fiber optic or microscopic visualization, and provide a post-operative recovery time that is substantially less than the recovery time that accompanies the corresponding open surgical approach.

Mold generally refers to the portion or portions of the present invention used to receive, constrain, shape and/or retain a flowable biomaterial in the course of delivering and curing the biomaterial in situ. A mold may include or rely upon natural tissues (such as the annular shell of an intervertebral disc) for at least a portion of its structure, conformation or function. The mold, in turn, is responsible, at least in part, for determining the position and final dimensions of the cured prosthetic implant. As such, its dimensions and other physical characteristics can be predetermined to provide an optimal combination of such properties as the ability to be delivered to a site using minimally invasive means, filled with biomaterial, prevent moisture contact, and optionally, then remain in place as or at the interface between cured biomaterial and natural tissue. In a particularly preferred embodiment the mold material can itself become integral to the body of the cured biomaterial.

The present mold preferably includes both a cavity for the receipt of biomaterial and two or more conduits to that cavity, although a single conduit is suitable for some applications. Some or all of the material used to form the mold will generally be retained in situ, in combination with the cured biomaterial, while some or all of the conduit will generally be removed upon completion of the method. Alternatively, the mold can be biodegradable or bioresorbable.

Biomaterial generally refers to a material that is capable of being introduced to the site of a joint and cured to provide desired physical-chemical properties in vivo. In a preferred embodiment the term will refer to a material that is capable of being introduced to a site within the body using minimally invasive means, and cured or otherwise modified in order to cause it to be retained in a desired position and configuration. Generally such biomaterials are flowable in their uncured form, meaning they are of sufficient viscosity to allow their delivery through a cannula of on the order of about 1 mm to about 6 mm inner diameter, and preferably of about 2 mm to about 3 mm inner diameter. Such biomaterials are also curable, meaning that they can be cured or otherwise modified, in situ, at the tissue site, in order to undergo a phase or chemical change sufficient to retain a desired position and configuration.

The present invention includes a method of implanting an intervertebral prosthesis in a disc located between a pair of adjacent vertebrae of a patient. Damaged or diseased nucleus pulpous is removed from the disc using minimally invasive techniques. The adjacent vertebrae are positioned in a lordotic condition. A mold adapted to contain a biomaterial is positioned between the adjacent vertebrae. A flowable biomaterial is delivered into the mold using minimally invasive techniques so that the adjacent vertebrae are in the lordotic condition. The flowable biomaterial is allowed to at least partially cure so that the adjacent vertebrae are in a lordotic-neutral position.

The step of positioning the pair of adjacent vertebrae in a lordotic condition may include positioning the patient in extension, displacing spinous processes of the adjacent vertebrae to a compressed configuration, suturing spinous processes of the adjacent vertebrae to a compressed configuration, and/or delivering the flowable biomaterial into the mold at sufficient pressure to distraction the adjacent vertebrae to a lordotic position.

In another embodiment, the step of positioning the pair of adjacent vertebrae in a lordotic condition includes providing the mold with an anterior portion and a posterior portion and delivering the flowable biomaterial to the anterior portion of the mold at a higher pressure than the pressure of the biomaterial in the posterior portion of the mold.

In another embodiment, the lordotic condition can be achieved by pressurizing the anterior chamber with a liquid and relaxing the tissue surrounding the intervertebral disc space. The biomaterial can then be delivered to the anterior portion of the mold at generally the same pressure as the posterior portion of the mold.

The step of providing the mold with an anterior portion and a posterior portion can be achieved by locating a partition inside the mold or providing a discrete anterior mold and a discrete posterior mold. In one embodiment, the discrete anterior and posterior molds can optionally be restrained relative to each other by mechanical fastener, a mesh bag, or a variety of other methods.

In another embodiment, the flowable biomaterial is delivered to the anterior and posterior portions of the mold at a pressure of about 5 atmospheres to about 10 atmospheres for anywhere between a few seconds and a few minutes. Thereafter, the pressure in the anterior portion is reduced and maintained at about 0.5 atmospheres to about 3 atmospheres, while the pressure in the posterior portion of the mold is reduced and maintained at about 0.5 atmospheres to about 2 atmospheres until the biomaterials are at least partially cured. The pressure can be reduced in the anterior and posterior portions of the mold simultaneously or at different times.

In another embodiment, the anterior portion of the mold is constructed with a first elasticity and the posterior portion of the mold with a second elasticity, wherein the first elasticity is greater than the second elasticity.

In one embodiment, the lordotic condition comprises about 25 degrees to about 30 degrees of lordosis, and more preferably about 10 degrees to about 15 degrees of lordosis, and most preferably about 15 degrees to about 20 degrees of lordosis.

### BRIEF DESCRIPTION OF THE SEVERAL VIEWS OF THE DRAWING

FIG. 1 is a schematic illustration of the forces that act on an intervertebral prosthesis during flexion of the spinal column.

FIG. 2 is a sectional view of the intervertebral prosthesis of FIG. 1.

FIG. 3 is a schematic illustration of an intervertebral prosthesis in accordance with the present invention.

FIG. 4 is a sectional view of the intervertebral prosthesis of FIG. 3 during the implant procedure.

FIG. 5 is a schematic illustration of a multi-chamber intervertebral prosthesis in accordance with the present invention.

FIG. 6 is a sectional view of the intervertebral prosthesis of FIG. 5 during the implant procedure.

FIG. 7 is a schematic illustration of an alternate multi-chamber intervertebral prosthesis in accordance with the present invention.

FIG. 8 is a sectional view of the intervertebral prosthesis of FIG. 7 during the implant procedure.

FIG. 9 is a schematic illustration of an alternate intervertebral prosthesis in accordance with the present invention.

FIG. 10 is a sectional view of the intervertebral prosthesis of FIG. 9 during the implant procedure.

FIG. 11 is a sectional view of an intervertebral disc with a preformed prosthesis in the posterior region and an inflatable prosthesis in the anterior region in accordance with the present invention.

FIG. 12 is a sectional view of an intervertebral disc with a preformed prosthesis in the anterior region and an inflatable prosthesis in the posterior region in accordance with the present invention.

FIG. 13 is a sectional view of an intervertebral disc with preformed prostheses in the anterior and posterior region in accordance with the present invention.

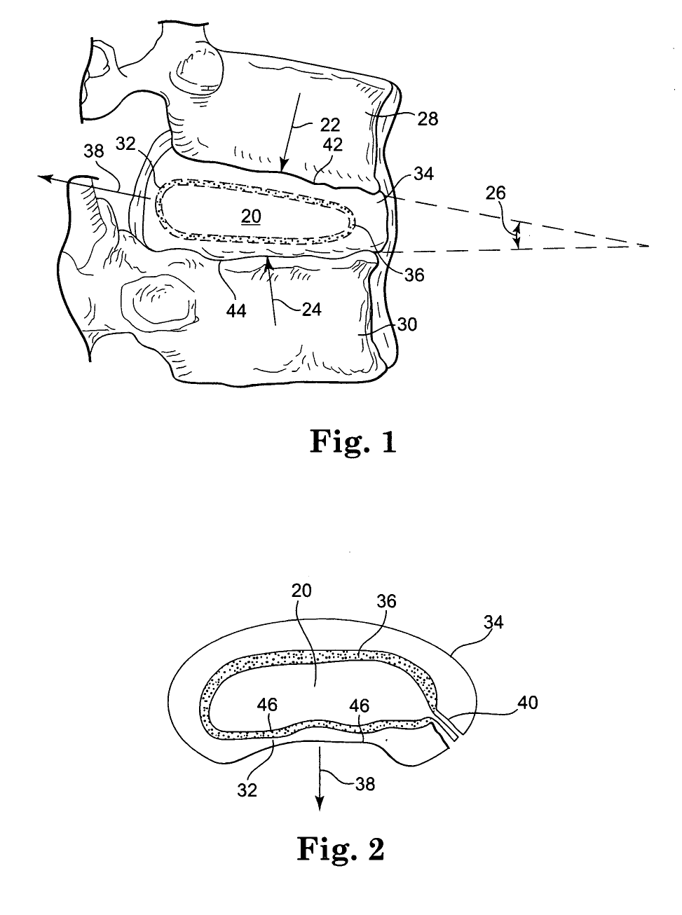

### DETAILED DESCRIPTION OF THE INVENTION

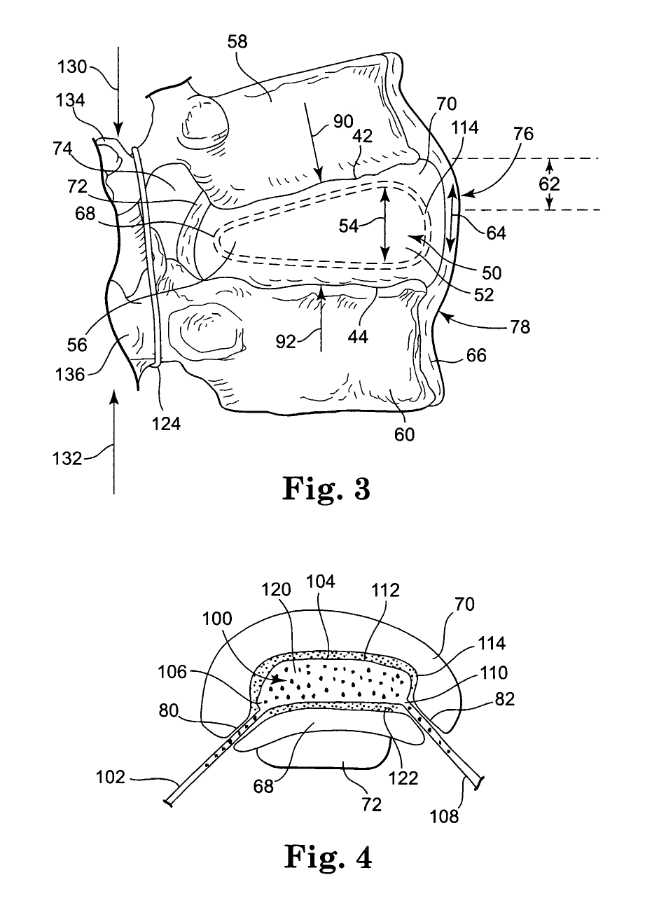

FIG. 3 is a schematic illustration of an intervertebral prosthesis 50 in accordance with the present invention. Anterior portion 52 of the intervertebral prosthesis 50 has a vertical height 54 greater than posterior portion 56 so that the adjacent vertebrae 58, 60 are maintained in lordotic condition 78 in accordance with the present invention.

The resting position of the lumbar spine at the L3-L4, L4-L5 and S1 vertebrae is normally in a lordotic position. In flexion, the lordosis is decreased or eliminated. In extension, the lordosis is increased. It is also possible to create lordosis by compressing the posterior portion 56 of annulus 70. This type of lordosis is undesirable because the posterior wall 68 may protrude into the spinal canal 74, and compressing the spinal cord or otherwise aggravating the patient's condition.

In the illustrated embodiment, the intervertebral prosthesis 50 creates a lordotic condition 78 in accordance with the present invention by applying a permanent anterior distraction 62. The anterior distraction 62 typically applies tension 64 to the anterior longitudinal ligament 66. The posterior wall 68 of the annulus 70 and the posterior longitudinal ligament 72 are preferably maintained in a neutral or undistracted condition. In an alternate embodiment, the posterior wall 68 and posterior longitudinal ligament 72 may be subject to some distraction or some compression.

As used herein, “lordotic condition” refers to primarily anterior distraction of a pair of adjacent vertebrae that does not cause symptomatic impingement of the spinal cord by the posterior portion of the intervertebral disc. The posterior wall 68 and posterior longitudinal ligament 72 of the intermediate intervertebral disc may be subject to some compression in the present lordotic configuration, as long as the patient is asymptomatic. The present lordotic condition is such that at least some lordosis is preferably maintained even during flexion of the intervertebral joint.

After implanting the present prosthesis, the lordotic condition becomes the neutral or resting position of the adjacent vertebrae. As used herein, “lordotic-neutral position” refers to an orientation of the effected adjacent vertebrae in a lordosis when the operative musculature is in a resting state.

The anterior longitudinal ligament 66 runs in the front (anterior) and vertically (longitudinal) attaching to the front of each vertebra 58, 60. The posterior longitudinal ligament 72 runs vertically behind (posterior) the vertebrae 58, 60 from the brain to the tailbone and inside the spinal canal 74. The ligamentum flavum (not shown) connects under the facet joints and forms a little curtain over the posterior opening between the vertebrae. This curtain can be pushed aside during surgery to allow the physician access to the spinal canal 74. Smaller ligaments that attach to the vertebral bodies 58, 60 to further safeguard the spine against bending too far in any direction join the three ligament systems.

As illustrated in FIG. 4, the preferred method includes one or more annulotomies 80, 82 in the annulus 70 laterally enough to avoid damage to the posterior longitudinal ligament 72 and the posterior wall 68 of the annulus 70. The present method preferably includes an MRI and a discogram preoperative assessment of the intervertebral disc. Interoperatively, a total nucleus removal (“TNR”) is performed. The annulus 70 is preferably preserved as much as possible.

After the central portion or nucleus pulpous 112 is substantially removed from the annulus 70, multi-lumen mold 100 is threaded through the annulotomies 80, 82 so that mold 104 is positioned within the annular cavity 114. First lumen 102 is fluidly coupled to mold 104 at location 106. Optional second lumen 108 is fluidly coupled to the mold 104 at location 110.

In a first embodiment in accordance with the present invention, the patient's body is configured in extension to create the lordotic condition 78 illustrated in FIG. 3. The patient may be restrained to the operating table to maintain the spine in extension.

The mold 104 is substantially filled with biomaterial 120. The biomaterial 120 can be delivered to the mold 104 through the first lumen 102, the second lumen 108, or some combination thereof. In one embodiment, the biomaterial 120 is delivered through the first lumen 102 while a vacuum or reduced pressure condition is applied to the second lumen 108. In an alternate embodiment, the mold 104 only has a single lumen 102. In the illustrated embodiment, a portion of the biomaterial 120 is drawn into the second lumen 108 once the mold 104 is fully inflated. After the biomaterial 120 is at least partially cured, the first and second lumens 102, 108 are cut, preferably flush with inner surface 122 of the annulus 70.

By maintaining the vertebrae 58, 60 in the lordotic condition 78, a greater quantity of the biomaterial 120 flows into the anterior portion 52 than in the posterior portion 56. The biomaterial 120 cures with a greater vertical height 54 in the anterior portion 52 than in the posterior portion 56, resulting in a permanent anterior distraction 62 that maintains the vertebrae 58, 60 in the lordotic condition 78 of the present invention.

In a second embodiment, forces 130, 132 are applied to the spinous processes 134 136 to create a compressed configuration. As used herein, “compressed configuration” refers to displacing spinous processes of adjacent vertebrae toward each other. The compressed configuration creates the lordotic condition 78 of the present invention.

The forces 130, 132 can optionally be created by wrapping suture material 124 around the spinous processes 134, 136. In one embodiment, the ends of the spinous processes 134, 136 are sutured together to create the lordotic condition 78 of FIG. 3. In one embodiment, the sutures 124 are cut following at least partial curing of the biomaterial 120. In another embodiment, the sutures 124 are bioresorbable so that by the time the patient recovers from the surgery, full motion is restored. In another embodiment, reference numeral 124 refers to an elastic material used to maintain tension and to allow flexion motion to occur. In one embodiment, the material 124 is easily removed following at least partial curing of the biomaterial 120, or at some later time after the surgical procedure.

Maintaining the vertebrae 58, 60 in the lordotic condition 78 causes forces 90, 92 to act against the prosthesis 50, thereby resisting extrusion towards the posterior wall 68. The angle of the end plates 42, 44 tends to urge the prosthesis 50 toward the anterior longitudinal ligament 66. During flexion the vertebrae 58, 60 are preferably still in the lordotic condition 78, such that the end plates 42, 44 still act to retain the intervertebral prosthesis 50 in the intervertebral disc space 76.

It is estimated that by maintaining the lordotic condition 78 of about 25 degrees to about 30 degrees, the expulsion force on the prosthesis 50, even during flexure, is not sufficient to extrude the prosthesis 50 through the posterior wall 68. For some patients the lordotic condition 78 is preferably about 10 degrees to about 15 degrees, and more preferably about 15 degrees to about 20 degrees, and most preferably about 20 degrees to about 30 degrees, depending on a number of factors such as for example the condition of the annulus, the size of the prosthesis required, the location of the annulotomy, and a variety of other factors.

In another embodiment, the mold 104 is formed so that inflation of the posterior portion 56 by the biomaterial 120 is constrained relative to the anterior portion 54. For example, the elasticity of the anterior portion 54 may be greater than the posterior portion. In one embodiment, the posterior portion is constructed from an inelastic material or is optionally surround by an inelastic material. In another embodiment, the anterior longitudinal ligament 66 can be relaxed, as discussed herein.

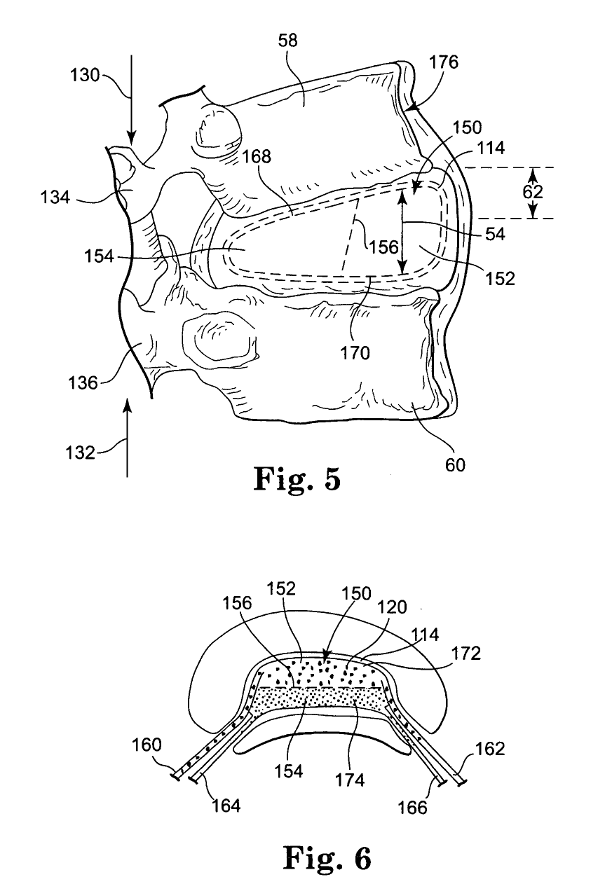

FIGS. 5 and 6 illustrate an alternate embodiment of the present method and apparatus. Mold 150 includes an anterior chamber 152 and a posterior chamber 154. The mold 150 is positioned in the annular cavity 114 as discussed above. In the illustrated embodiment, the mold 150 includes a partition 156 that separates the anterior chamber 152 from the posterior chamber 154. In the illustrated embodiment, the partition 156 is preferably a rigid or semi-rigid material so that the pressure of the biomaterial 172 in the anterior chamber 152 can be greater than the pressure of the biomaterial 174 in the posterior chamber 154.

The anterior chamber 152 includes first and second lumens 160, 162 while the posterior chamber 154 includes first and second lumens 164, 166. Although the embodiment of FIG. 6 illustrate two lumens for each chamber 152, 154, it is possible for the mold 150 to include a single lumen with each chamber.

The pressure and quantity of biomaterials 172, 174 in the respective chambers 152, 154 can be independently controlled to permit the vertebrae 58, 60 to be positioned in lordotic condition 176.

In one embodiment, the biomaterials 172, 174 are the same materials. In another embodiment, the biomaterials 172, 174 are different materials. The biomaterials 172, 174 can be delivered simultaneously or sequentially. In one embodiment, the biomaterial 172 is delivered first. After the biomaterial 172 is at least partially cured, the biomaterial 174 is delivered. In another embodiment, the biomaterial 174 is delivered first. After the biomaterial 174 is at least partially cured, the biomaterial 172 is delivered.

In another embodiment, the wall 168 of the posterior chamber 154 has a greater wall thickness than wall thickness of the wall 170 of the anterior chamber 152. The greater thickness of the wall 168 restricts expansion of the posterior chamber 154, while the lesser thickness of the wall 170 permits the anterior chamber 152 to achieve the greater vertical height 54.

In anther embodiment, the wall 168 proximate posterior chamber 154 is constructed from a material with less elasticity than the wall 170 proximate the anterior chamber 152. In yet another embodiment, tension members can be wrapped around or embedded in the wall 168 to constrain expansion of the posterior chamber 154.

In another embodiment, the chambers 152, 154 are filled with biomaterials 172, 174, respectively at a pressure of about 5 atmospheres to about 10 atmospheres for anywhere between a few seconds and a few minutes. Thereafter, the pressure in the anterior chamber 152 is reduced and maintained at about 0.5 atmospheres to about 3 atmospheres, while the pressure in the posterior chamber 154 is reduced and maintained at about 0.5 atmospheres to about 2 atmospheres until the biomaterials 172, 174 are at least partially cured. The pressure can be reduced in the anterior and posterior chambers 152, 154 simultaneously or at different times. For example, the pressure in the anterior chamber 152 may be maintained for a longer period than the posterior chamber 154. As discussed in connection with FIG. 3, the greater vertical height 54 of the anterior chamber 152 applies a permanent anterior distraction 62 that creates the desired lordotic condition 176.

In one embodiment, the lordotic condition 176 of the vertebrae 58, 60 can be created simply by controlling the flow of biomaterials 172, 174 to the chambers 152, 154 of the mold 150. In an alternate embodiment, the method may include positioning the patient in a lordotic condition 176 and/or applying forces 130, 132 to the spinous processes 134, 136, such as discussed above.

In another embodiment, the anterior chamber 152 can be pressurized with a fixed volume of saline or a liquid contrast medium to the level anticipated during delivery of the biomaterial 172. Images of the intervertebral disc space are optionally taken at various pressures to measure the distraction of the adjacent vertebrate. After a period of time, such as about a few seconds to about five minutes, the tissue surrounding the intervertebral disc space, in particular the anterior longitudinal ligament 66 (see FIG. 3), relaxes causing the pressure measured in the anterior chamber 152 to drop. Additional saline or contrast medium is then introduced into the anterior chamber 152 to increase the pressure in the intervertebral disc space to the prior level. The tissue surrounding the intervertebral disc space again relaxes.

By repeating this procedure several times, the lordotic position 176 is more easily achieved. In one embodiment, the lordotic position 176 can be achieved by delivering the biomaterials 172, 174 at generally the same pressure. The method of relaxing the tissue surrounding the intervertebral disc space can be used with any of the embodiments disclosed herein. In another embodiment, a separate evaluation mold is used to perform the relaxation cycles of the tissue surrounding the intervertebral disc space. Once the relaxation cycles are completed, the evaluation mold is removed and the mold 150 is inserted.

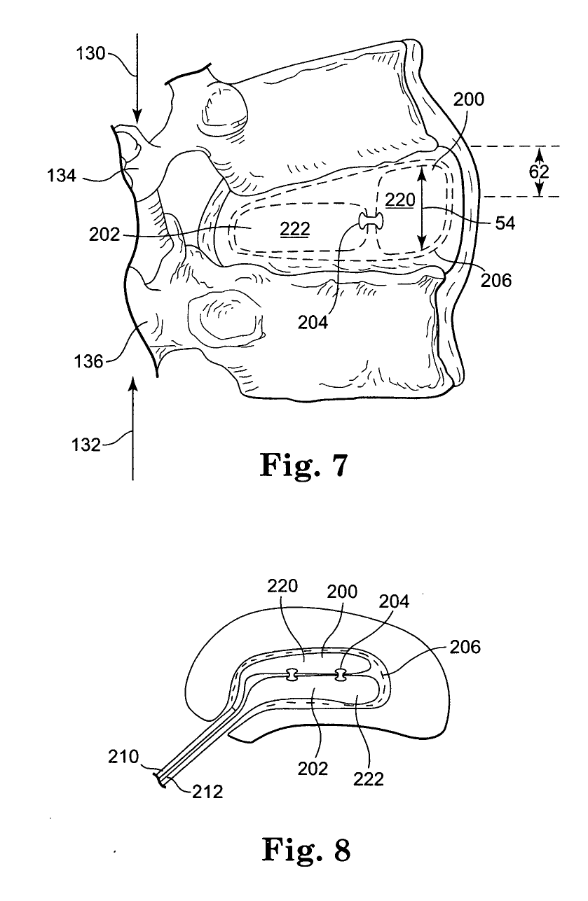

FIGS. 7 and 8 illustrate an alternate apparatus comprising a discrete anterior mold 200 and a discrete posterior mold 202. The anterior mold 200 and posterior mold 202 can be securely connected to each other using a variety of techniques. In one embodiment, the anterior mold 200 is securely connected to the posterior mold 202 by one or more mechanical fasteners 204. In an alternate embodiment, a mesh bag 206 or other containment vessel surrounds both the anterior mold 200 and posterior mold 202.

As illustrated in FIG. 8, lumen 210 is fluidly coupled to the anterior mold 200 and lumen 212 is fluidly coupled to the posterior mold 202. In an alternate embodiment, one or more of the molds 200, 202 may include secondary lumens, such as illustrated in FIGS. 4 and 6.

In one embodiment, mold 200 is an evaluation mold used to perform the relaxation cycles of the tissue surrounding the intervertebral disc space discussed above. Once the relaxation cycles are completed, the evaluation mold 200 is removed and the molds 200, 202 are inserted.

In one embodiment, the mold 200 is constructed of a material and/or thickness having greater elasticity than the mold 202. In another embodiment, the mold 200 is configured to create the greater vertical height 54 along the anterior side of the vertebrae 58, 60, and hence, the permanent anterior distraction 62 of the present lordotic condition. In another embodiment, different biomaterials 220, 222 are delivered to the molds 200, 202, respectively. The discrete molds 200, 202 permit the respective biomaterials 220, 222 to be different or the same and/or to be delivered at different pressures.

As discussed in connection with FIGS. 5 and 6, the patient can also be positioned in a lordotic condition and/or forces 130, 132 can be applied to the spinous processes 134, 136 in order to achieve the illustrated lordotic condition of the vertebrae 58, 60 during delivery of the biomaterial 220, 222.

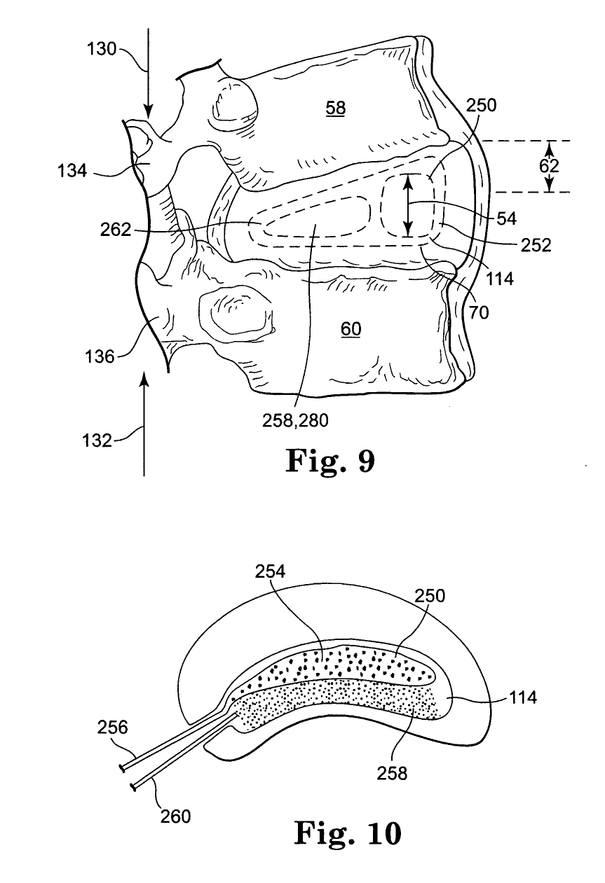

FIGS. 9 and 10 illustrate another embodiment of the present method and apparatus. Mold 250 is located in anterior portion 252 of the annular cavity 114. Biomaterial 254 is delivered to the mold 250 through lumen 256. Biomaterial 258 is delivered through lumen 260 directly into posterior region 262 of the annular chamber 114, without the use of a mold. The annulus 70 serves as the mold for the biomaterial 258.

The mold 250 provides the anterior distraction 62 necessary to achieve the vertical height 54. The biomaterial 258 helps to secure the mold 250 in the anterior portion 252 of the annulus 70. The biomaterials 254, 258 can be the same or different material.

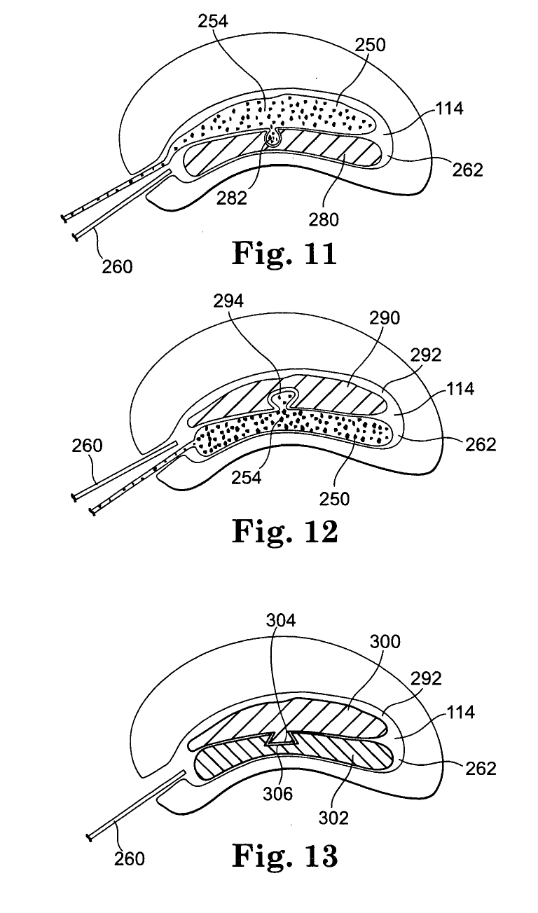

In an alternate embodiment illustrated in FIG. 11, a preformed prosthesis 280 is delivered through lumen 260 directly into posterior region 262 of the annular chamber 114. The preformed prosthesis 280 can optionally be constructed from two or more sections that are assembled in situ. The position of the prosthesis 280 within the annular chamber 114 relative to the mold 250 is shown schematically in FIG. 9 without the interlocking relationship. In the illustrated embodiment, the prosthesis 280 includes one or more structures 282 that engage with the mold 250. In the preferred embodiment, the biomaterial 254 forces a portion of the mold 250 into recess 282 in the prosthesis 280 to form an interlocking relationship.

As discussed in connection with FIGS. 5 and 6, the patient can also be positioned in a lordotic condition and/or forces 130, 132 can be applied to the spinous processes 134, 136 in order to achieve the illustrated a lordotic condition of the vertebrae 58, 60 during delivery of the biomaterials 254, 258.

FIG. 12 illustrates preformed prosthesis 290 delivered through lumen 260 directly into anterior region 292 of the annular chamber 114. The mold 250 is located in the posterior region 262. The size and shape of the prosthesis 290 relative to the mold 250 creates the lordotic condition. In the illustrated embodiment, the prosthesis 290 includes one or more structures 294 that engage with the mold 250. In the preferred embodiment, the biomaterial 254 forces a portion of the mold 250 into recess 294 in the prosthesis 290 to form an interlocking relationship.

FIG. 13 illustrates two or more preformed prostheses 300, 302 delivered through lumen 260 into the annular chamber 114. The prosthesis 300 is located in the anterior region 292, while the prosthesis 302 is located in the posterior region 262. In the illustrated embodiment, the prostheses 300, 302 preferably have features 304, 306 that form an interlocking relationship within the annular chamber 114. The size and shape of the prosthesis 300 relative to the prosthesis 302 creates the lordotic condition.

The molds of the present invention can also be used for evaluating the nuclectomy or the annulus and for imaging the annulus prior to delivery of the biomaterial(s). Disclosure related to evaluating the nuclectomy or the annulus, use of an evaluation mold, and delivering the biomaterial are found in U.S. patent application Ser. No. 10/984,493, entitled “Multi-Sage Biomaterial Injection System for Spinal Implants, which is incorporated by reference. Various implant procedures and biomaterials related to intervertebral disc replacement suitable for use with the present method and apparatus are disclosed in U.S. Pat. No. 5,556,429 (Felt); U.S. Pat. No. 6,306,177 (Felt, et al.); U.S. Pat. No. 6,248,131 (Felt, et al.); U.S. Pat. No. 5,795,353 (Felt); U.S. Pat. No. 6,079,868 (Rydell); U.S. Pat. No. 6,443,988 (Felt, et al.); U.S. Pat. No. 6,140,452 (Felt, et al.); U.S. Pat. No. 5,888,220 (Felt, et al.); U.S. Pat. No. 6,224,630 (Bao, et al.), and U.S. patent application Ser. Nos. 10/365,868 and 10/365,842, all of which are hereby incorporated by reference.

Various delivery catheters and catheter holders suitable for performing the present method are disclosed in commonly assigned U.S. patent application Ser. No. ______, entitled Catheter Holder for Spinal Implants, filed on the same date herewith (Attorney Docket No. 321296), which is hereby incorporated by reference. The molds of the present invention can also be secured to the annulus using any of the methods and devices disclosed in commonly assigned U.S. Patent application Serial No. entitled Multi-Lumen Mold For Intervertebral Prosthesis And Method Of Using Same, filed on the same date herewith (Attorney Docket No. 321297), which is hereby incorporated by reference.

Patents and patent applications disclosed herein, including those cited in the Background of the Invention, are hereby incorporated by reference. Other embodiments of the invention are possible. Many of the features of the various embodiments can be combined with features from other embodiments. It is to be understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

What is claimed is:

1. A method of implanting an intervertebral prosthesis in a disc located between a pair of adjacent vertebrae of a patient, the method comprising the steps of:
using minimally invasive techniques to remove damaged or diseased nucleus pulpous from the disc;
positioning the pair of adjacent vertebrae in a lordotic condition;
positioning a mold adapted to contain a biomaterial between the adjacent vertebrae;
delivering a flowable biomaterial into the mold using minimally invasive techniques so that the adjacent vertebrae are in the lordotic condition; and
allowing the flowable biomaterial to at least partially cure so that the adjacent vertebrae are in a lordotic-neutral position.

2. The method of claim 1 wherein the step of positioning the pair of adjacent vertebrae in a lordotic condition comprises positioning the patient in extension.

3. The method of claim 1 wherein the step of positioning the pair of adjacent vertebrae in a lordotic condition comprises the step of displacing spinous processes of the adjacent vertebrae to a compressed configuration.

4. The method of claim 1 wherein the step of positioning the pair of adjacent vertebrae in a lordotic condition comprises the step of suturing spinous processes of the adjacent vertebrae to a compressed configuration.

5. The method of claim 1 wherein the step of positioning the pair of adjacent vertebrae in a lordotic condition comprises delivering the flowable biomaterial into the mold at sufficient pressure to distraction the adjacent vertebrae to the lordotic condition.

6. The method of claim 1 wherein the step of positioning the pair of adjacent vertebrae in a lordotic condition comprises the steps of:
providing the mold with an anterior portion and a posterior portion; and
delivering the flowable biomaterial to the anterior portion of the mold at a higher pressure than the pressure of the biomaterial in the posterior portion of the mold.

7. The method of claim 6 wherein the step of providing the mold with an anterior portion and a posterior portion comprises locating a partition inside the mold.

8. The method of claim 6 wherein the step of providing the mold with an anterior portion and a posterior portion comprises the step of providing a discrete anterior mold restrained relative to a discrete posterior mold.

9. The method of claim 6 comprising the steps of:
delivering the flowable biomaterial to an anterior portion of the mold at a pressure of about 5 atmospheres to about 10 atmospheres; and
delivering the flowable biomaterial to a posterior portion of the mold at a pressure of about 2 atmospheres to about 3 atmospheres.

10. The method of claim 1 wherein the step of positioning the pair of adjacent vertebrae in a lordotic condition comprises the steps of:
providing the mold with an anterior portion and a posterior portion; and
delivering the flowable biomaterial to the anterior portion of the mold;
allowing the flowable biomaterial to at least partially cure; and
delivering a biomaterial to the posterior portion of the mold.

11. The method of claim 1 wherein the step of delivering a flowable biomaterial into the mold comprising the steps of:
delivering a first biomaterial at a first pressure to an anterior portion of the mold; and
delivering a second biomaterial at a second pressure to a posterior portion of the mold, wherein the first pressure is greater than the second pressure.

12. The method of claim 11 comprising the step of constructing the anterior portion and posterior portion of the mold as first and second discrete molds.

13. The method of claim 1 wherein the step of delivering a flowable biomaterial into the mold comprising the steps of:
constructing an anterior portion of the mold with a first elasticity; and
constructing a posterior portion of the mold with a second elasticity, wherein the first elasticity is greater than the second elasticity.

14. The method of claim 13 comprising the step of constructing the anterior portion and posterior portion of the mold as first and second discrete molds.

15. The method of claim 1 wherein the step of positioning a mold between the adjacent vertebrae comprises the steps of:
positioning an anterior mold in an anterior region between the adjacent vertebrae;
positioning a posterior mold in a posterior region between the adjacent vertebrae;
delivering the biomaterial to the anterior and posterior molds.

16. The method of claim 15 comprising the steps of:
delivering a first biomaterial to the anterior mold; and
delivering a second biomaterial to the posterior mold.

17. The method of claim 15 comprising the steps of:
delivering the biomaterial to the anterior mold at a first pressure; and
delivering the biomaterial to the posterior mold at a second pressure lower than the first pressure.

18. The method of claim 15 comprising the steps of:
delivering the flowable biomaterial to the anterior mold;
allowing the flowable biomaterial to at least partially cure; and
delivering a biomaterial to the posterior mold.

19. The method of claim 15 comprising the steps of:
delivering the flowable biomaterial to the posterior mold;
allowing the flowable biomaterial to at least partially cure; and
delivering a biomaterial to the anterior mold.

20. The method of claim 15 comprising the step of attaching the anterior mold to the posterior mold using mechanical fasteners.

21. The method of claim 15 comprising the step of retaining the anterior mold and the posterior mold in a mesh bag.

22. The method of claim 1 wherein the step of positioning a mold between the adjacent vertebrae comprises the steps of:
positioning an anterior mold in an anterior region between the adjacent vertebrae;
positioning one or more preformed prosthesis in a posterior region between the adjacent vertebrae; and
delivering the biomaterial to the anterior mold.

23. The method of claim 22 comprising the step of interlocking the anterior mold with the preformed posterior prosthesis.

24. The method of claim 1 comprising the steps of:
delivering a liquid under pressure to the mold sufficient to distract the intervertebral disc space;
holding the volume of liquid in the mold constant for a period of time; and
adding additional liquid to the mold when the pressure in the mold drops to a predetermined level.

25. The method of claim 24 comprising repeating the steps of delivering, holding and adding additional liquid a plurality of cycles.

26. The method of claim 24 comprising the step of delivering the liquid under pressure to an anterior region of the mold.

27. The method of claim 1 wherein the lordotic condition comprises about 25 degrees to about 30 degrees of lordosis.

28. The method of claim 1 wherein the lordotic condition comprises about 10 degrees to about 15 degrees of lordosis.

29. The method of claim 1 wherein the lordotic condition comprises about 15 degrees to about 20 degrees of lordosis.

30. The method of claim 1 comprising the steps of:
delivering the flowable biomaterial to an anterior portion of the mold at a pressure of about 5 atmospheres to about 10 atmospheres; and
delivering the flowable biomaterial to a posterior portion of the mold at a pressure of about 2 atmospheres to about 3 atmospheres.

31. A method of implanting an intervertebral prosthesis in a disc located between a pair of adjacent vertebrae of a patient, the method comprising the steps of:
using minimally invasive techniques to remove damaged or diseased nucleus pulpous from the disc;
positioning the pair of adjacent vertebrae in a lordotic condition;
positioning one or more preformed prosthesis in a posterior region between the adjacent vertebrae; and
positioning one or more preformed prosthesis in a anterior region between the adjacent vertebrae.
